(19) United States
(12) Patent Application Publication
(10) Pub. No.: US 2002/0150596 A1
(43) Pub. Date: Oct. 17, 2002

(54) Ointment having micro bubbles containing a liquid and methods

(76) Inventors: Donald R. Carter, Englewood, CO (US); Stephen M. Mitchell, Brighton, CO (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 10/108,798

(22) Filed: Mar. 27, 2002

Related U.S. Application Data
(63) Continuation-in-part of application No. 09/464,293, filed on Dec. 15, 1999.
Application No. 09/464,293 is a continuation-in-part of application No. 09/316,426, filed on May 21, 1999, now abandoned.

(51) Int. Cl. A61K 38/28; A61K 31/56

(52) U.S. Cl. 424/400; 514/3; 514/169; 514/509

(57) ABSTRACT

A topical ointment comprises a base material which itself may comprise mineral oil and polyethylene glycol. Encapsulated within the base material are micro bubbles that contain a liquid. The ointment may further include additional substances encapsulated therein. Application of the topical ointment to a body area provides for the moisturizing and the slow delivery of the additional substance to the applied area. The ointment interacts with the body heat emitted from the body area and dissolves and provides a slow rate of delivery of the materials encapsulated therein.

### CROSS REFERENCE TO RELATED APPLICATIONS

This application is a continuation-in-part application of U.S. patent application Ser. No. 09/464,293, filed Dec. 15, 1999, which is a continuation-in-part of U.S. patent application Ser. No. 09/316,426 filed on May 21, 1999, the complete disclosures of which are herein incorporated by reference.

### FIELD OF THE INVENTION

The present invention relates to a topical ointment and more particularly to a topical ointment which may act as a moisturizer and delivery system of substances to bodily areas upon which it is applied.

### BACKGROUND OF THE INVENTION

A number of methods and apparatus exist for delivering drugs and other pharmaceuticals to parts of the human body. In oral delivery of the drug, the active agent enters the bloodstream by being absorbed in the lining of the stomach. Another drug delivery system is through direct injection via a needle into the bloodstream. Other possible drug delivery systems include the administration of a suppository, endotracheal administration, and eye dropping administration. Disadvantages of the above described methods may include lack of absorption of the drug through stomach lining, the pain experienced from injections, and the inability to deliver measured amounts of the drugs over predetermined periods of time.

Another method of delivering a drug or pharmaceutical is through application of a substance carrying the drugs or pharmaceuticals to a mucosal surface of the body. The drug or other pharmaceutical composition may be mixed with a petroleum-based jelly and the combination topically applied to a mucosal surface, such as those in the nasal passageway. As is documented, the placement of petroleum-based products in the airways, may have certain medical side effects.

### SUMMARY OF THE INVENTION

The invention provides topical ointments that supply, among other features, moisturization upon application to various body surfaces. In situations where the ointment includes additional substances such medications, the ointment may further provide for the controlled delivery of these substances to surfaces upon which the ointment has been applied. Upon application of the topical ointment to different body cavities, the delivery of the additional substances to the surfaces within the cavity may be occur at a slow release rate.

The topical ointments may include a base material that is both plasticized and gelatinized. A plasticized base is a base that has chemical that increase the stability of its ingredients, and a gelatinized base is a base that has been transformed from a liquid into a gel. In one particular embodiment, the base may comprise a combination of mineral oil and polyethylene glycol. Encapsulated within the base material may be gas bubbles that include a quantity of a liquid, such as an aqueous solution. Examples of aqueous solutions that may be encapsulated include a mixture of NaCl with sterile H₂O, and bacteriostatic water.

Also encapsulated within the base material may be one or more additional substances which may include, but is not limited to, various drugs or pharmaceutical substance such as antibiotics, steroids, aromatic oils, nitroglycerine, painkillers, nicotine and humalog insulin.

In the manufacture process for the topical ointment, a quantity of the base material is placed in a device which acts to mix, beat, whip, blend and/or stir the materials contained therein. The material is mixed at a certain rate to control the size of the gas bubbles formed therein. The lower the rate at which the mixing process may occur the smaller the gas bubbles created in the base material. The percentages of the aqueous solution versus the additional materials encapsulated may be varied to control the concentration or the additional substances to be delivered. Further, the percentage of the base material versus the other elements may also be changed to vary the delivery rate.

Once the mixing process is begun, the aqueous solution may be added. The mixing process may act to encapsulate the aqueous solution within the gas bubbles created in the base material. The aqueous solution may be added to the base material in portions during the mixing process until each portion is encapsulated within the base material.

Additional substances may also be encapsulated during a further mixing process. Portions of the additional substance may be added to the base material and aqueous solution during the mixing process until the entire amount is encapsulated.

In use, topical ointment may be applied to various body surfaces. When the ointment comes in contact with the body surface, body heat may act to dissolve the base material providing for the release to the surface of the aqueous solution and other substances encapsulated therein. The rate the base material dissolves may be related to the proximity of that portion of the ointment to the body surface as well as other environmental factors.

As the base material dissolves, the materials encapsulated within the base material are delivered to the body surface. This continuous exposure act to hydrate the affected area as well as provide exposure to the additional substances. Through this exposure medications may be absorbed into the blood stream.

In one specific aspect, the ointment may comprise a formulation of PEG, mineral oil and bacteriostatic normal saline. The ointment may utilize a high-grade pharmaceutical mineral oil having a specific gravity that allows it to bind under complex gradients, i.e., heat and shear, with a low molecular weight polyethylene glycol. The mineral oil and PEG form a complex matrix of alkylene bonding. Such a formulation results in essentially no residual mineral oil, as it becomes part of the matrix and is incorporated into the complex bonding of the carbons. This creates an irreversible compound, causing gelatinization and plastization of the base. Bacteriostatic components and buffered normal saline may be added to this base. The normal saline is encapsulated in micro bubbles within the plasticized base to create a safe, non-petroleum, time released gel. The chemical moiety does not have mineral oil in its finished stated. As such, it does not revert back to its mineral oil form because its has been plasticized. Further, microencapsulation of the saline provides a time release lasting approximately 8 to 12 hours with a single application. The ointment is also pH balanced to the nasal mucosa.

### BRIEF DESCRIPTION OF THE DRAWING

FIG. 1 is a partial cross sectional view of an ointment according to the invention.

### DESCRIPTION OF THE SPECIFIC EMBODIMENTS

Disclosed herein is a topical ointment which may be applied to various parts of the human body, and through exposure to body heat, provide delivery of moisturizing agents as well as other substances. In general, the topical ointment comprises a base material containing small bubbles which is combined with an aqueous solution or other liquid which may provide hydration upon application. Also included may be additional substances such as medications or aromatic oils, which when encapsulated within the topical ointment provide for a slow release of the substance to a body surface.

The topical ointment includes a base material that may be both plasticized and gelatinized. Examples of base materials that may be used include those constructed of mineral oil and polyalkylene (e.g., polyethylene glycol or PEG), as well as Eucerin™, Aquaphor™, A&D Ointment™, Noxema™, petrolatum™, Nivia™, Vaseline™, Aveeno™cream or ointment, bees wax, paraffin, Lanolin™, emulsifying ointments or creams, hydrophilic creams or ointments, and the like. One exemplary base is a plasticized base that comprises about one percent to about nine percent, and more preferably from about four percent to about six percent of a low-molecular weight polyethylene glycol and about 91 percent to about 99 percent, and more preferably from about 94 percent to about 96 percent mineral oil. The PEG may have a molecular weight in the range from about 400 to about 600, and may be a combination of different molecular weights. For example, the PEG may comprise about 20 percent of a 400 molecular weight PEG and about 80 percent of a 600 molecular weight PEG. As another example, the PEG may comprise about 50 percent of a 400 molecular weight PEG and about 50 percent of a 600 molecular weight PEG. Further, it will be appreciated that other combinations are possible that are able to produce a PEG in the 400 to about 600 molecular weight range. Such a polymer is soluble in mineral oil above about ninety degrees Fahrenheit, which is close to its melting point. When the solution is cooled below ninety degrees Fahrenheit, the polymer precipitates and causes gelatinization. One example of such a plasticized base is manufactured by Professional Compounding Associates of Houston Tex.

The base material also includes micro bubbles that are encapsulated by the base material. These micro bubbles are at least partially filled with a liquid. In this way, as the base material slowly melts, the liquid is slowly released as described hereinafter. The micro bubbles may have a size in the range from about 10 microns to about 150 microns, and more preferably from about 25 microns to about 50 microns. Further, the base material may include about 2,000 to about 25,000 bubbles per cubic inch. Still further, the base material may have a viscosity in the range from about 6 to about 14, and more preferably of about 10. In another aspect, the base material may have a pH of about 5.7. Depending on whether the base includes any additives, the base material may have a pH in the range from about 4.0 to about 6.8.

A variety of liquids may be contained within the micro bubbles. For example, one liquid that may be encapsulated in the topical ointment may be an aqueous solution. The aqueous solution may comprise approximately 0.9% NaCl, and the remaining portion sterile H₂O. This aqueous solution may include other compounds which the ointment may deliver upon application to a body surface.

Other and/or additional substances that may be encapsulated in the base material include a large variety of drugs, oils, dietary supplements and other medications. These substances may be nontoxic, water soluble or miscible materials that are capable of being absorbed through the skin.

One type of substance that may be encapsulated in the topical ointment are antibiotics. The antibiotic may include: Gentamycin, Tetracycline, Erythromycin, and Cephalexin.

Another substance that may be encapsulated in the ointment includes steroids. Such steroids may include: Prednisone, Dexamethasone, and Prednisolone.

Other substances that may be encapsulated in the base material are aromatic oils. These oils may be employed in treatments such as aroma therapy. The oils may include: Eucalyptus Oil, Rosemary Oil, Pine Needle Oil, Tea Tree Oil, Wintergreen Oil, Peppermint Oil, Spearmint Oil, Camphor Oil, Sage Oil, Jojoba Oil, Cinnamon Oil, Anise Oil, Lemon Oil, Lime Oil, Orange Oil, Clove Oil, Almond Oil, White Pine Oil, Cardamon Oil and Cedar Leaf oil.

Still yet other substances that may be encapsulated within the base material are dietary supplements. These supplements may include: various vitamins, iron, potassium, calcium, potassium, magnesium, copper, zinc and the like.

Other substances that may be encapsulated include various medications. These medications may include: humalog insulin, anti-nausea medication (Prochloroperazine or Promethazine), smoking prevention medication (nicotine resin), painkilling medication (codeine, hyrocodone), nitro-glycerin, and the like.

In one embodiment, the formulation for the topical ointment may comprise approximately 50-80% by weight of the base material. Additionally, the formulation may comprise approximately 0-50% by weight of the aqueous solution. In formulations where an additional substance is included, 0-30% by weight of the additional substance may be included. With regards to the various substances described above, the composition of the topical ointment is such that one or more different substances (for example antibiotics and aromatic oils) may be encapsulated therein.

The ointments of the invention may be prepared by continuous or batch processes. As in preparing conventional emulsions, shear forces may be applied to the components by use of mixers, blenders, homogenizers, mills, impingement surfaces, ultrasound, shaking or vibration. To get the desired micro bubbles within the base material, the mixing shear may be at low levels. The higher the shear the larger the micro bubbles.

The base material may comprise a variety of plasticized and gelatinized materials as previously described. In some cases, such base materials may be purchased commercially. For example, one type of base may comprise a petroleum base. Alternatively, the base material may be manufactured from starting materials. For example, one type of base material may be formed by combining mineral oil and PEG in amounts previously described. The mineral oil and PEG may be mixed using a variety of techniques, such as by use of a standard inverse rotational blade mixer, or by a homogenizing unit. Examples of such equipment include, for example, a BioMixer having inverse rotational blades (which may include a grinding mill for adding excipients) or a Baum homogenizing unit having inverse rotational blades. Such a homogenizer sucks the mixture into a homogenizing unit with air and then pushes the mixture out in a repeating fashion. The mixing blades may be operated in the range from about 2,000 rpm to about 25,000 rpm. For instance, a standard inverse rotational mixer may be operated from about 8 to 12 hours at a speed of about 2,000 rpm. However, increased operating times may be used for lower speeds and vice versa. As another example, the homogenizer may be operated for about 15 minutes at a speed of about 25,000 rpm. Increased times may be used for decreased speeds and vice versa.

Once mixed, the base material is heated above its boiling point, typically above 20 to about 30 degrees C. The applied heat is used to form alkaline bonds. The base is mixed at the heated temperature until plasticized. For instance, mixing may occur for about 30 minutes to more than about 2 hours. Preferably, a constant shear is applied at a constant temperature to form the alkaline bonds. The base material is then permitted to cool to room temperature where the base material gelatinizes.

The ointment is formed from the base material by forming micro bubbles in the base material and incorporating a liquid, such as an aqueous solution and/or any additional substances within the micro bubbles. To form the micro bubbles, a quantity of the base material may be placed in a mixing device and a whipping or mixing process may then performed on the base material. The rate at which the base material is mixed or whipped is related to the size of the encapsulations created within the material. As mentioned above, the faster and shorter the mixing process, the larger the encapsulations will be. The size of the encapsulations is relevant because this is related to the rate at which the aqueous solution and the additional substances are delivered to the body area to which the ointment is applied. For example, the ointment may be produced with micro bubbles having a mean size in the range from about 0.01 mm to about 3 mm. When placed onto living tissue that has a temperature of about 95 to about 100 degrees Fahrenheit, the ointment will melt at a rate in the range from about 1/24 to about ⅙ of total volume per hour, releasing about 1/24 to about ⅙ of the liquid per hour. The rate of melting may vary depending upon the temperature of the tissue and its location. For example, when placed in the nasal cavity, the user's breathing convects cooler air over the ointment to reduce its melting rate.

The liquid may be added to the base material before, during or after the micro bubbles are formed to hydrate the base material. Any of the mixing techniques described herein may be used when adding the liquid. If the liquid is added before mixing, the liquid osmotically passes through the base material and into the micro bubbles as they are formed. If the liquid is added after the micro bubbles are formed, additional mixing is performed to move the liquid into the base material where is enters into the micro bubbles. The entire amount of the liquid may be added at once, or it may be added in separate portions to avoid any loss of solution due to splashing or other effects.

One type of liquid that may be added is an aqueous solution, such as a saline solution and/or bacteriostatic water. Such an aqueous solution may be added in an amount based on weight to volume. For example, the ratio may be about two-thirds weight to about one-third volume, e.g., for about 600 grams of the base material, about 300 ml of an aqueous solution may be added. One particular type of aqueous solution that may be added comprises a 0.9 percent sodium chloride solution to which ahs been added about 9 mls of Benzyl alcohol per about 1 gram of sodium chloride solution. This solution creates a bacteriostatic effect as it is released onto tissue. For instance, when placed in the nasal mucosa, the nasal passage is kept generally sterile as the solution is released onto the nasal mucosa. Further, the solution also acts as a moisturizer to keep the nasal mucosa moist. For other areas of the body, the moisture helps to reduce healing times and prevent infection. In other embodiments, the added liquid may comprise benzyl alcohol, alone or in combination with saline, mineral oil, propylene glycol, water or the like.

Upon the addition of the liquid, the base material mixture may be monitored to detect when the desired amount aqueous solution has been encapsulated therein. One sign that encapsulation is complete is the lack of standing liquids in the container employed for mixing.

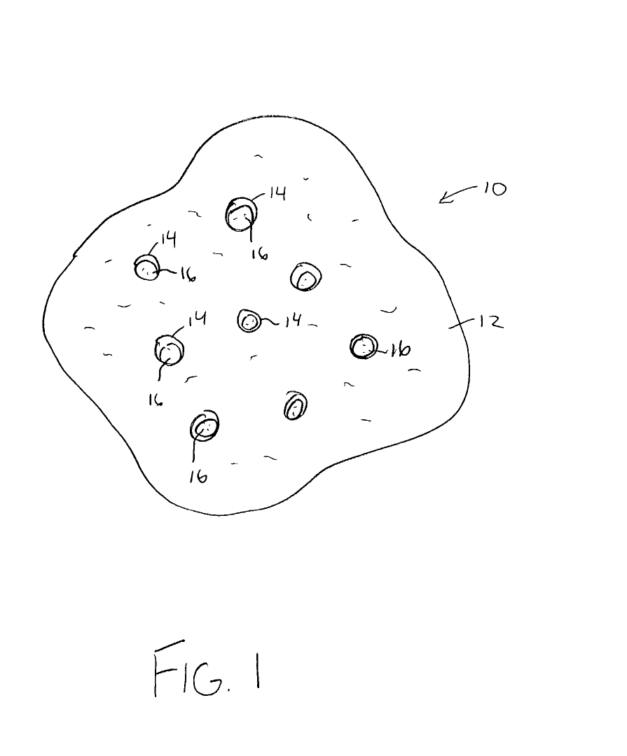

One example of an ointment 10 that has been produced using such a technique is illustrated in FIG. 1. Ointment 10 includes a base material 12 containing a plurality of bubbles 14 that are encapsulated by base material 12. Within bubbles 14 is an amount of a liquid 16. Liquid 16 may partially or completely fill each bubble 14.

If additional substances are to be added, the mixing of the base material continues after encapsulation of the initial liquid, e.g., an aqueous solution. A quantity of the additional substances is then added. If loss of the additional substance may be a problem (i.e., due to splashing, etc.) the additional substance may be added in smaller portions. It should be noted that the above process may be performed at room temperature. However, the ambient temperature should not to exceed the melting temperature of the base material.

The percentages of the base material, aqueous solution, and additional substances used during the formulation process can be varied depending upon the type of additional material employed and the desired rate or delivery of the encapsulated materials. For example, if the percentage of base material versus aqueous solution and additional substances is increased, the delivery rate will be increased. Conversely, if the percentage is decreased, the rate of delivery will increase.

In use, the ointment can be applied directly to various body surfaces. The base material may have a melting temperature of approximately 90 degrees Fahrenheit. Upon application to a body surface, the body heat, which may be approximately 95 to 100 degrees Fahrenheit will begin to dissolve the bonds of the polyethylene glycol in the base material, which in turn releases an amount of the encapsulated aqueous solution and additional substance, if encapsulated, to the body area. The combination of the aqueous solution and mineral oil act as a moisturizing element for the exposed area.

The slow release of the aqueous solution and additional substances is made possible by the fact that due to the chemical make-up of the polyethylene glycol, it will retain its structure and bond the mineral oil and hold the encapsulated material until a melting temperature is reached. When an amount of the ointment is applied to a body area, the portion of the ointment in contact with the skin will have a higher temperature than the amount which is further from the skin. In practice the portion of the ointment next to the skin will dissolve delivering its encapsulated material to the area while the portions away from the skin will remain cooler and thus retaining the encapsulated materials.

For example, the ointment has special applicability for use within the nasal cavity upon the nasal mucosa. Upon application, the portion in contact with the skin will dissolve and deliver the encapsulated materials. Conversely, the portion of the ointment exposed to the air will stay in the gelatinous state due to, in most cases, the lower ambient temperature of the air. The cooler temperatures are also due to evaporative cooling effects which are caused by the movement of air through the nasal cavity. This provides the benefit that the entire amount of ointment applied to the bodily surface does not dissolve and expose the entire amount of the encapsulated material to the area at once. All of the dissolved materials may be absorbed by the skin and the delivery of the additional substance to the bloodstream may occur. The ointment may be applied to other mucosal surfaces (vagina, rectum) but the rate of delivery may be greater due to the lack of moving air.

### EXAMPLE 1

Plasticized base 500 g (66.67% by weight)

Bacteriostatic H₂O 193.75 g (25.83% by weight)

Gentamycin 56.25 g (7.5% by weight)

In the manufacture of one formulation of the topical ointment, 500 grams of Plasticized base may be placed in the mixing apparatus, such as a table top mixer. The mixer is started at a low sheer rate and the Plasticized base is mixed until the desired encapsulations are formed. With 500 g of the material, it may take approximately 1 hour. The next step is to add all or a portion of the 193.75 cc's of bacteriostatic H₂O to the Plasticized base. The mixing process is monitored to note when the desired amount of the bacteriostatic H₂O is encapsulated within the Plasticized base. With the above recited amounts, the encapsulation of the bacteriostatic water may take approximately 8-12 hours.

When the encapsulation process of the bacteriostatic water is complete, the mixing process is continued, and the antibiotic, Gentamycin, is added. In this formulation, three milligrams per gram of Gentamycin are included. This equates to 56.25 grams of the antibiotic, which when fully encapsulated after the mixing process generates seven hundred and fifty grams of the topical ointment. Encapsulation of the Gentamycin may take approximately 3-4 hours.

Upon completion of the mixing process, the ointment is ready for topical use. If the ointment is to be applied in the nasal cavity, a typical dosage may be 0.1 g, or 100 mg, per nostril. Upon application, the total amount may dissolve in approximately 8-12 hour period. The dosage of the antibiotic in the ointment is approximately the same amount of the drug as if it had been prescribed orally. In situations where a higher dosage of an additional substance is called for, the amount of aqueous solution may be varied to compensate.

The foregoing description of the present invention has been presented for purposes of illustration and description. Furthermore, the description is not intended to limit the invention to the form disclosed herein. Consequently, variations and modifications commensurate with the above teachings, and the skill or knowledge of the relevant are, within the scope of the present invention. The embodiments described hereinabove are further intended to explain best modes known for practicing the invention and to enable others skilled in the art to utilize the invention in such, or other, embodiments and with various modifications required by the particular applications or uses of the present invention. It is intended that the appended claims be construed to include alternative embodiments to the extent permitted by the prior art.

What is claimed is:

1. A topical ointment comprising:
a base material that is configured to melt at a rate in the range from about 1/24 to about ⅙ of total volume per hour when placed on living tissue that is at a temperature in the range from about 90 degrees Fahrenheit to about 105 degrees Fahrenheit, wherein the base material includes a plurality of gas bubbles that are encapsulated by the base material; and
a liquid contained within the gas bubbles.

2. The topical ointment of claim 1, wherein the base material includes about 2,000 to about 25,000 gas bubbles per cubic inch of the base material.

3. The topical ointment of claim 1, wherein the gas bubbles have a mean size in the range from about 10 microns to about 150 microns.

4. The topical ointment of claim 1, wherein the liquid comprises bacteriostatic water.

5. The topical ointment of claim 1, wherein the liquid comprises NaCl and sterile H₂O.

6. The topical ointment of claim 1, further comprising an additional substance that is selected from a group consisting of: an antibiotic, steroid, humalog insulin, nitro-glycerin, aromatic oils, anti-nausea medication, pain killers, dietary supplements, and smoking cessation medication.

7. The topical ointment of claim 1, wherein the base material is plasticized and gelatinized.

8. The topical ointment of claim 1, wherein the base material has a viscosity in the range from about 6 to about 14, and a pH in the range from about 4.0 to about 6.8.

9. The topical ointment of claim 1, wherein the base material comprises about 91% to about 98% mineral oil and about 9% to about 2% polyethylene glycol.

10. A method of preparing a topical ointment comprising the steps of:
mixing a plasticized and gelatinized base material until gas bubbles exist within the base, wherein the gas bubbles have a mean size in the range from about 10 microns to about 150 microns, and wherein the gas bubbles have a density of about 2,000 to about 25,000 gas bubbles per cubic inch of the base material, and
adding a liquid into the base, wherein the liquid enters into the gas bubbles and is encapsulated in the base material.

11. The method of claim 10, wherein the liquid is added prior to the mixing step, and wherein the liquid osmotically moves into the gas bubbles formed by the mixing.

12. The method of claim 10, wherein the liquid is added after the gas bubbles are formed, and further comprising performing a subsequent mixing step to incorporate the liquid into the gas bubbles.

13. The method of claim 10, further comprising adding at least one additional substance to the base and mixing the base material until the at least one additional substance is also contained within the gas bubbles.

14. The method of claim 13, wherein the at least one additional substance is selected from a group consisting of: an antibiotic, steroid, humalog insulin, nitro-glycerin, aromatic oils, anti-nausea medication, pain killers, dietary supplements, and smoking cessation medication.

15. The method of claim 10, wherein the base material is plasticized and gelatinized.

16. The method of claim 10, wherein the base material has a viscosity in the range from about 6 to about 14, and is configured to melt at a rate in the range from about 1/24 to about ⅙ of total volume per hour when placed on living tissue that is at a temperature in the range from about 90 degrees Fahrenheit to about 105 degrees Fahrenheit.

17. The method of claim 10, wherein the base material comprises about 91% to about 98% mineral oil and about 9% to about 2% polyethylene glycol.

18. A method for delivering of a substance to living tissue, the method comprising:
providing an ointment comprising a base material that is configured to melt at a rate in the range from about 1/24 to about ⅙ total volume per hour when placed on living tissue that is at a temperature in the range from about 90 degrees Fahrenheit to about 105 degrees Fahrenheit, wherein the base material includes a plurality of gas bubbles that are encapsulated by the base material, and wherein a liquid is contained within the gas bubbles; and
applying the ointment to living tissue to permit the base material to melt over time due to the heat of the living tissue and to slowly release the liquid to the living tissue.

19. The method of claim 18, wherein the living tissue comprises the nasal mucosa.

20. The method of claim 18, wherein the based material includes about 2,000 to about 25,000 gas bubbles per cubic inch, and wherein the gas bubbles have a mean size in the range from about 10 microns to about 150 microns.

21. The method of claim 18, wherein the liquid comprises bacteriostatic water.

22. The method of claim 18, wherein the liquid comprises NaCl and sterile H₂O.

23. The method of claim 18, wherein the ointment further includes an additional substance that is selected from a group consisting of an antibiotic, steroid, humalog insulin, nitro-glycerin, aromatic oils, anti-nausea medication, pain killers, dietary supplements, and smoking cessation medication that is included within the gas bubbles and that is released to the living tissue as the base material melts.

24. The method of claim 18, wherein the base material is plasticized and gelatinized.

25. The method of claim 1, wherein the base material has a viscosity in the range from about 6 to about 14.

25. The method of claim 18, wherein the base material comprises about 91% to about 98% mineral oil and about 9% to about 2% polyethylene glycol.
